(19) United States
(12) Patent Application Publication
(10) Pub. No.: US 2009/0156903 A1
(43) Pub. Date: Jun. 18, 2009

(54) ARTICULATING HOOK ELEVATOR AND ARTHROSCOPIC METHOD FOR USING SAME

(76) Inventors: Gregory A. Guederian, Naples, FL (US); Scott P. Steinmann, Rochester, MN (US)

Correspondence Address: DICKSTEIN SHAPIRO LLP, 1825 EYE STREET NW, Washington, DC, 20006-5403, US

(21) Appl. No.: 12/329,401

(22) Filed: Dec. 5, 2008

Related U.S. Application Data
(60) Provisional application No. 61/014,254, filed on Dec. 17, 2007.

(51) Int. Cl. A61B 1/32

(52) U.S. Cl. 600/204

(57) ABSTRACT

An articulating hook elevator for manipulating tissue during arthroscopic procedures. The articulating hook elevator comprises a shaft, a proximal end, and a distal end provided with an articulating paddle. The hook may be actuated by a switch and can articulate into a standard tip for traditional manipulation of tissue, or into a rotated or articulated position. In this manner, effective manipulation and retraction of tissue from the surgical site without tissue collapse may be achieved, allowing a surgeon to better visualize the internal condition of the arthroscopic site and speed up the overall procedure.

### CROSS-REFERENCE TO RELATED APPLICATIONS

This application claims the benefit of U.S. Provisional Application No. 61/014,254, filed Dec. 17, 2007, the entire disclosure of which is incorporated by reference herein.

### FIELD OF THE INVENTION

The present invention relates to arthroscopic surgical methods and instruments and, more specifically, to an articulating hook elevator for arthroscopy.

### BACKGROUND OF THE INVENTION

Arthroscopic surgery involves the insertion of an arthroscope into a joint region, such as the knee, elbow or shoulder, to allow a surgeon to view the internal condition of the joint. Examples of such arthroscopic procedures are partial meniscectomies and ligament reconstructions in the knee, shoulder acromioplasties and rotator cuff debridements, and elbow synovectomies.

During arthroscopic surgery, a small incision is made in the skin covering the arthroscopic site or joint so that surgical instruments may be placed in the joint and manipulated through arthroscopic visualization. A very small incision is highly desirable as it has an obvious cosmetic advantage, and low complication rates with a very low incidence of infection.

Because only a very small incision is made during arthroscopic surgery, it is often difficult to grab small regions of tissue and to subsequently apply a desired tension on the tissue within the joint capsule, either in a direction toward or away from the arthroscopic portal. In addition, it is also difficult to handle instruments within the joint capsule, where visibility and access to the structures of the joint capsule is minimal.

Accordingly, a need exists for a surgical instrument that allows improved handling of instrumentation within a joint capsule, for example the elbow capsule, during athroscopic surgery. A need also exists for a surgical instrument that is stable during elbow arthroscopy and that allows the secure lifting and/or retracting orientation desired by the surgeon, without accidental slipping or shifting and with minimal soft tissue edema to the patient.

### SUMMARY OF THE INVENTION

The present invention provides an articulating hook elevator having a shaft, a proximal end, and a distal end provided with an articulating hook. The hook is configured to allow secure engagement and retraction of anatomical structures (such as neurovascular structures) during arthroscopic surgery. The hook may be actuated by a switch and can articulate into a standard tip for traditional manipulation of tissue, or into a rotated or articulated position.

Other features and advantages of the present invention will become apparent from the following description of the invention, which refers to the accompanying drawings.

### BRIEF DESCRIPTION OF THE DRAWINGS

FIG. 1 is a perspective view of an articulating hook elevator according to an embodiment of the present invention.

FIG. 2 is a top view of the articulating hook elevator of FIG. 1.

FIG. 3 is a cross-sectional view of the articulating hook elevator of FIG. 1 and in an articulating position.

FIG. 4(a) is an enlarged view of the distal end of the articulating hook elevator of FIG. 1 and in a non-articulating position.

FIG. 4(b) is an enlarged view of the switching mechanism of the articulating hook elevator of FIG. 1 and in a non-articulating position.

FIG. 5 is a partial cross-sectional view of the articulating hook elevator of FIG. 1 and in a non-articulating position.

FIG. 6(a) is enlarged view of the distal end of the articulating hook elevator of FIG. 1 and in an articulating position.

FIG. 6(b) is an enlarged view of the switching mechanism of the articulating hook elevator of FIG. 1 and in an articulating position.

FIG. 7 is a partial cross-sectional view of the articulating hook elevator of FIG. 1 and in an articulating position.

FIG. 8 is a lateral view of an elbow joint undergoing arthroscopy and with the articulating hook elevator of the present invention.

### DETAILED DESCRIPTION OF THE PREFERRED EMBODIMENTS

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The present invention provides an articulating hook elevator having a shaft, a proximal end, and a distal end provided with an articulating hook. The hook is configured to allow secure engagement and retraction of anatomical structures (such as neurovascular structures) during arthroscopic surgery.

The hook may be actuated by a switch and can articulate into a standard tip for traditional manipulation of tissue, or into a rotated or articulated position. The switch mechanism (in the form of a mechanical cam) actuates the tip of an articulating hook elevator to a rotated or “bent tip” position, in the manner described in U.S. Patent Application Publication No. 2005/0203345, entitled “Articulating Paddle Elevator and Arthroscopic Method for Using Same,” the disclosure of which is hereby incorporated by reference in its entirety.

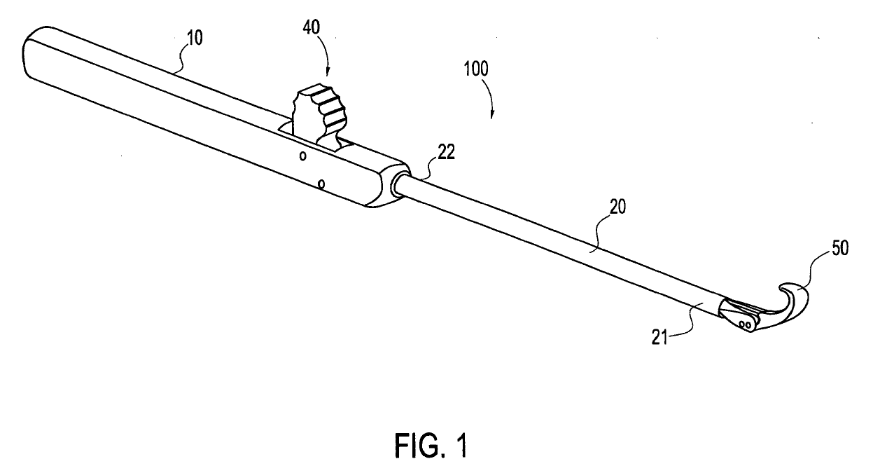

Referring now to the drawings, where like elements are designated by like reference numerals, FIGS. 1-7 illustrate an articulating hook elevator 100 of the present invention. The articulating hook elevator 100 of FIGS. 1-7 may be used to manipulate and palpate tissue during arthroscopic surgery (for example, elbow arthroscopy) and to lift and/or retract tissue while maintaining capsular distention without damaging adjacent structures such as neurovascular structures, for example. The hook of the articulating instrument allows engagement and retraction of neurovascular structures without the risk of sliding off and subsequent injury of such structures during surgery. Thus, the hook elevator of the present invention allows more controlled arthroscopic engagement and secure lifting and/or retracting orientation desired by the surgeon, without accidental slipping or shifting and with minimal soft tissue edema to the patient.

As illustrated in FIG. 1, the hook elevator 100 includes a shaft 20 provided in the shape of a cylinder and having a distal end 21 and a proximal end 22. A handle 10 is disposed at the proximal end 22 of the shaft 20, and an actuating tip or hook 50 is located at the distal end 21 of the shaft 20. The actuating hook 50 has a configuration that allows it to securely engage (to hook) additional tissue structures (such as neurovascular structures, for example) and to retract these structures, without accidental slipping or shifting and with minimal tissue edema to the patient. The actuating hook 50 may be additionally used for manipulating and palpating tissue.

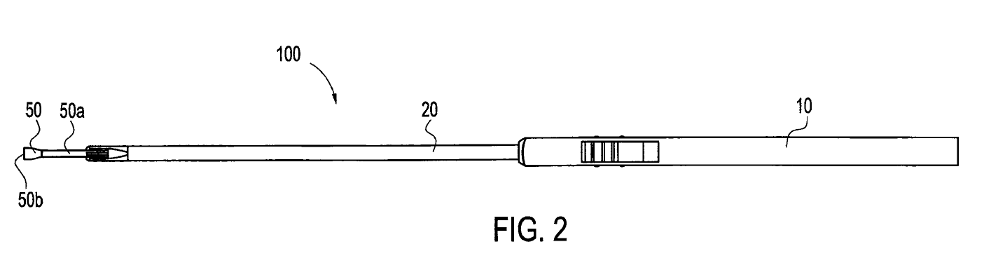

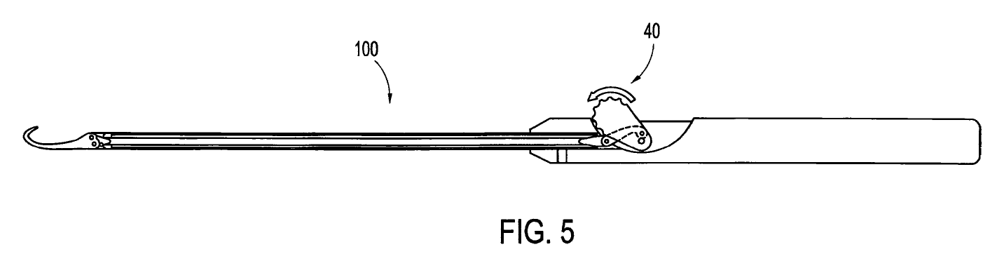

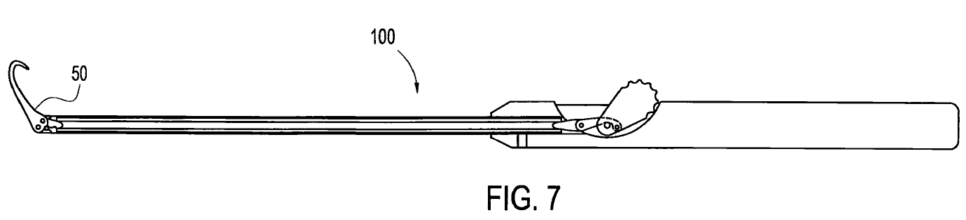

As illustrated in FIGS. 2, 3 and 4(a), for example, the articulating or actuating hook 50 comprises a main body region 50a (in the form of a paddle) that is integral to a curved, hooked region 50b positioned at the most distal end of the main body region 50a. When the instrument is in a non-articulating position (such as the one illustrated in FIG. 5, for example), a longitudinal axis (including a main paddle surface) of the main body region 50a is about parallel to the longitudinal axis of the instrument. When the instrument is in an articulating position (such as the one illustrated in FIG. 7, for example), the longitudinal axis (including the main paddle surface) of the main body region 50a is about non-parallel to the longitudinal axis of the instrument.

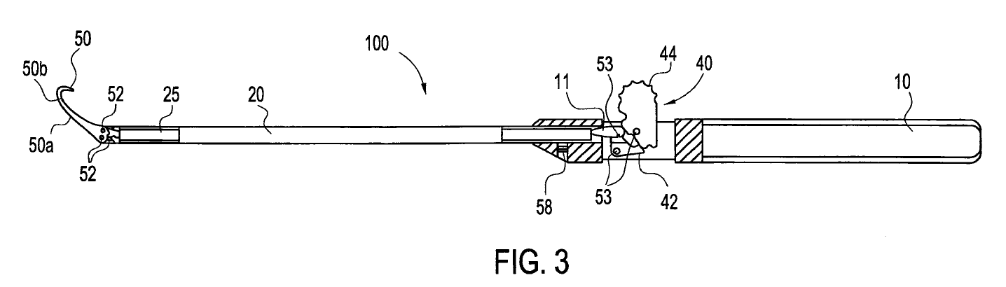

An actuator 40 is located at the proximal end of the shaft 20 and connected to the handle 10. The actuator 40 comprises a lever or thumb trigger 44, a link 42, a cam 11 and an actuator 25 connected to the link 42 and the trigger 44. As shown in FIG. 3, the actuator 40 also comprises a set screw 58 and a plurality of handle pins 53. The actuator 40 is designed to cause the actuating hook 50 of the hook elevator 100 to be angled, for example at about 40°, when actuated. The actuating hook 50 is connected to actuator 25 by a plurality of pins 52 (FIG. 3).

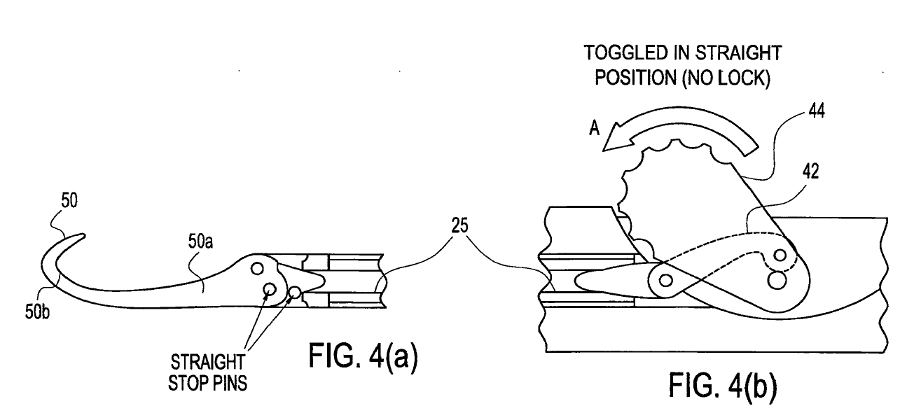

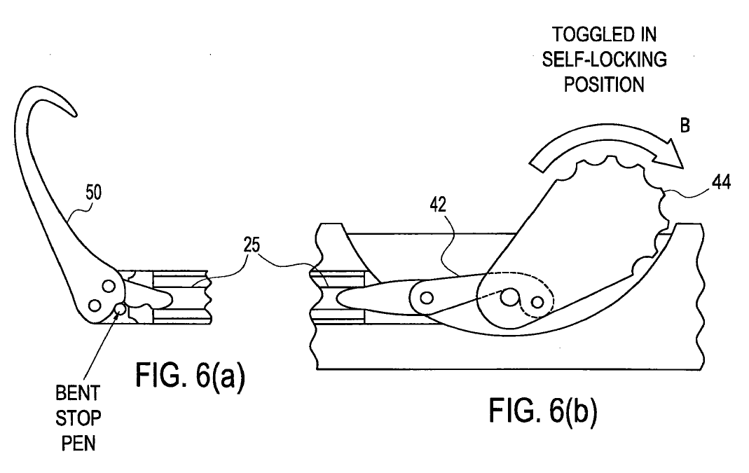

FIGS. 4-7 illustrate the mechanics of the articulating hook elevator 100 of the present invention. FIG. 5 illustrates the articulating hook elevator 100 disposed in the straight or “unlocked” position, while FIG. 7 illustrates the hook elevator 100 disposed in the articulating or “bent tip” position and locked. FIGS. 4(a)-(b) and FIGS. 6(a)-(b) are enlarged views of the articulating hook and switch mechanism corresponding to the straight and articulating positions of the articulating hook elevator 100 of FIGS. 5 and 7, respectively.

The articulating hook 50, which rotates to an angle of about 40° (in the embodiment shown in FIGS. 3 and 7) to about 120°, is connected to the actuator 40 which comprises the lever or trigger 44, link 42, cam 11 and actuator 25 connected to the hook 50 at the distal end. The switch mechanism 40 is mechanically connected to the hook 50 and, when actuated, causes the hook 50 to rotate to a 40° position, for example, as shown in FIGS. 3 and 7. In the straight position shown in FIG. 5, the trigger 44 is pushed in the direction of arrow A (FIG. 4(b)) and disposed in a front or “unlocked” position. This causes the actuator 25 to be biased towards the distal end of articulating hook elevator 100.

FIG. 7 illustrates the articulating hook elevator 100 in the “bent tip” or “locked” position, in which the articulating hook 50 is disposed in a 40° position. To rotate the articulating hook to the 40° position, trigger 44 is pushed backward in the direction of arrow B of FIG. 6(b). Trigger 44 includes a cam 11 which pushes the actuator 25 backward or towards the proximal end of the actuator when trigger 44 is moved backward. This mechanical action causes the hook 50 to rotate 40° as shown.

The articulating hook elevator 100 of the present invention described above with reference to FIGS. 1-7 may be employed in various surgical medical procedures for manipulating body tissue and neurovascular structures during surgical procedures. For example, the articulating hook elevator 100 may be employed in endoscopic and arthroscopic procedures, including but not limited to elbow arthroscopy, knee arthroscopy, shoulder arthroscopy, and other arthroscopic procedures that require handling and manipulation (lifting and/or retracting) of tissue while maintaining capsular distention without damaging adjacent structures such as neurovascular structures, for example.

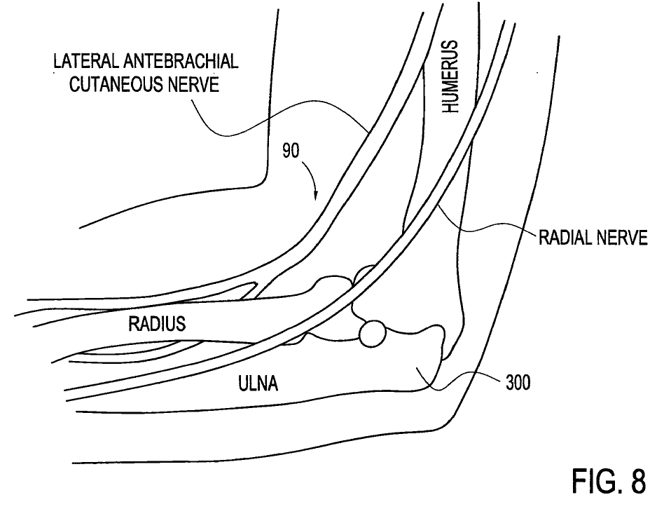

To better illustrate an exemplary surgical procedure conducted with the articulating hook elevator 100 of the present invention, reference is now made to FIG. 8, which illustrate a side schematic view of a surgical site 90 of elbow joint 300. A surgeon advances articulating hook elevator 100 in the “straight” or closed configuration into elbow joint 300, as shown in FIG. 8, optionally through a small cannula or portal, for example. The “straight” configuration allows the surgeon to gently insert instrument 100 into the elbow capsule and out of adjacent vital neurovascular structures, such as the brachial artery, the median nerve and the radial nerve, for example.

Once the articulating hook elevator 100 is inserted into the elbow joint, the surgeon then articulates hook 50 to a desired angle, for example to approximately 40° or 120°. The surgeon may also gradually increase or decrease the angle of the hook (for example, from a first position to a second position), as desired and in accordance with the characteristics of the surgical site. The hook of the articulating instrument 100 allows engagement and retraction of neurovascular structures (such as the ulnar nerve, for example) without the risk of sliding off and subsequent injury of such structures during surgery. The hook elevator of the present invention allows a more controlled arthroscopic engagement and secure lifting and/or retracting orientation desired by the surgeon, without accidental slipping or shifting and with minimal soft tissue edema to the patient.

Although the present invention has been described in relation to particular embodiments, many other variations and modifications and other uses will become apparent to those skilled in the art.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

What is claimed is:

1. An articulating hook elevator, comprising:
a cannulated shaft having a distal end and a proximal end, and a longitudinal axis;
an articulating hook located at the distal end of the cannulated shaft, the articulating hook being configured to move relative to the longitudinal axis of the cannulated shaft from a first position to a second position; and
an actuator assembly located at the proximal end of the cannulated shaft and operatively connected to the articulating hook.

2. The articulating hook elevator of claim 1, wherein the articulating hook includes a body extending in a first orientation and a most distal curved region extending in a second orientation which is different from the first orientation.

3. The articulating hook elevator of claim 1, wherein the body in integral to the most distal curved region.

4. The articulating hook elevator of claim 2, wherein in the first position, the body of the articulating hook forms with the longitudinal axis of the cannulated shaft an angle of about 0°.

5. The articulating hook elevator of claim 4, wherein in the second position, the body of the articulating hook forms with the longitudinal axis of the cannulated shaft an angle of about 5° to about 175°.

6. The articulating hook elevator of claim 5, wherein the body of the articulating hook forms with the longitudinal axis of the cannulated shaft an angle of about 80°.

7. The articulating hook elevator of claim 1, wherein the actuator assembly comprises an actuator operatively connected to the articulating hook, and a switch mechanism connected to the actuator.

8. The articulating hook elevator of claim 7, wherein the switch mechanism comprises a trigger, a link and a cam.

9. The articulating hook elevator of claim 1, wherein the elevator is used in elbow arthroscopy.

10. The articulating hook elevator of claim 1, wherein the actuator assembly rotates the articulating hook relative to the longitudinal axis of the cannulated shaft.

11. A surgical instrument for arthroscopy, comprising:
a shaft having a proximal end, a distal end and a longitudinal axis, the shaft comprising an actuator disposed coaxially within the shaft;
a hook located at the distal end of the shaft and coupled to the actuator, the hook being capable of rotating relative to the longitudinal axis of the shaft, the hook comprising a curved portion and a paddle adjacent the curved portion, the paddle being integral to the curved portion; and
a switch assembly located at the proximal end of the shaft and coupled with the actuator.

12. The surgical instrument of claim 11, wherein the switch assembly comprises a cam mechanism that controls the movement of the actuator by manually pushing a trigger of the switch assembly.

13. The surgical instrument of claim 11, wherein the paddle has an elliptical configuration.

14. A method of endoscopically manipulating tissue, comprising the steps of:
providing an articulating hook elevator comprising a cannulated shaft having a distal end and a proximal end; an articulating hook located at the distal end of the cannulated shaft, the articulating hook being capable of being moved relative to a longitudinal axis of the cannulated shaft from a first position to a second position; and a switch assembly located at the proximal end of the cannulated shaft and operatively connected to the articulating hook;
positioning the articulating hook elevator in the proximity of tissue to be manipulated at a surgical site;
advancing the articulating hook elevator in the first position through the tissue; and
actuating the switch assembly to move the articulating hook from the first position to the second position and to manipulate tissue at the surgical site.

15. The method of claim 14, wherein, in the first position, a body of the articulating hook forms with the longitudinal axis of the cannulated shaft an angle of about 0°.

16. The method of claim 14, wherein, in the second position, a body of the articulating paddle forms with the longitudinal axis of the cannulated shaft an angle of about 5° to about 175°.

17. The method of claim 14, wherein the surgical site comprises elbow capsular tissue.
